(19) United States
(12) Patent Application Publication
(10) Pub. No.: US 2014/0172446 A1
(43) Pub. Date: Jun. 19, 2014

(54) SYSTEM AND METHOD FOR THE TRACEABILITY OF A SURGICAL INSTRUMENTATION SYSTEM

(71) Applicant: Pierre Dumouchel, Asnieres Sur Seine (FR)

(72) Inventor: Pierre Dumouchel, Asnieres Sur Seine (FR)

(73) Assignee: SAFE ORTHOPAEDICS, Eragny Sur Oise (FR)

(21) Appl. No.: 14/122,806

(22) PCT Filed: May 22, 2012
(86) PCT No.: PCT/FR2012/051139

§ 371 Date: Mar. 5, 2014

(30) Foreign Application Priority Data
Jun. 1, 2011 (FR) 11/01703

(51) Int. Cl. G06F 19/00; A61B 19/00

(52) U.S. Cl. CPC G06F 19/3406; A61B 19/44
USPC 705/2

(57) ABSTRACT

A surgical instrumentation system has a single-use instrument kit for fitting implants and at least one sterile implant kit, each of the kits comprising an identifier, at least one of the kits contains a unique identifier, as well as a medium for recording additional identifiers, one of the additional identifiers being a patient code, collected when the kit is used, and at least one other additional identifier being the identifier of at least one complementary kit comprising an identifier transferable to the additional-identifier recording medium, when the complementary kit is used. A method for implementing such a system is also described.

### BACKGROUND

1. Field of the Invention

The present invention concerns the field of the management of surgical instruments and implants for single use, and particular orthopaedic prostheses and the associated instrumentation.

2. Prior Art

The American patent application US 2003/0182299 is known in the prior art, describing a system consisting of a plurality of sterile instrument kits intended for fitting implants not included in the kits. Each kit is marked with a unique identifier.

The patent application WO 03081379 is also known, describing a solution for managing a unique kit containing surgical instruments and intra-ocular implants.

This solution does not make it possible to ensure traceability of surgical systems where the instrumentation is provided in a first kit and some of the implants in another kit distinct from the first.

The documents DE 4420707, EP 1855264, WO 2005/027767 and WO 2011/035277 are also known, describing traceability labels and methods applicable in the medical field.

The international application WO 2008/043921 is also known, describing a method and monitoring of medical products in which the product and/or its packaging is marked and identified with a first reference. This first reference is stored in a first file. The product is delivered in a packaged form for its subsequent use by the customer, for treating a patient listed in a second file. The references of the product are detected automatically and remotely when it is delivered. These references are stored in a third stock management file of the customer at the time of an operation. The solution of the prior art makes provision for automatically detecting the product being brought out of stock, inserting the references of the product in the second file corresponding to the patient for which it is—or will be—used. A fourth file is then established, comprising the partial references coming from the patient file and the determined references of the product for tracing, and this fourth file is automatically or semi-automatically transmitted to the supplier of the product.

### Drawbacks of the Solutions of the Prior Art

The solutions proposed in the prior art have several technical drawbacks.

First, they necessarily require access to a digital information system for processing and using the identification data.

For use in an operating theatre, use of a data processing means is not appropriate, and this leads to recording of the information before or after the information. Deferring this recording is a source of errors or omissions, which is prejudicial to confidence in the traceability of the products and instruments used.

Secondly, the solutions of the prior art make provision, in the case of the application WO 2008/043921, for the recording in a system of information identifying the patient. This constraint comes up against the rules governing the confidentiality of medical data and management thereof, even if the description of this application evokes secret management of the identification of the patient.

The solution proposed in the application US 2003/0182299 on the contrary provides no link between the patient and the surgical equipment and concerns only the management of the stocks by a computer system.

None of the solutions of the prior art therefore makes it possible to ensure complete traceability by the hospital completely complying with the rules relating to the anonymity of the patient. This omission is a problem since the persons affected by this management of surgical instrumentation systems are not all doctors bound by medical confidentiality.

Moreover, none of the solutions of the prior art makes it possible to ensure traceability of all the instruments and implants used for a given patient.

### SUMMARY OF THE INVENTION

The purpose of the present invention is to remedy these drawbacks by proposing a system simple in its instrumentation and able to be used in the strict context of the management of medico-legal data.

To this end, the invention concerns, in its most general sense, a surgical instrumentation system consisting of a single-use instrument kit for fitting an implant and at least one sterile implant kit, each of said kits comprising an identifier, characterised in that at least one of said kits contains a unique identifier, as well as a medium for recording additional identifiers,

one of said additional identifiers being a patient code, collected when said kit is used,

at least one other additional identifier being the identifier of at least one complementary kit comprising an identifier transferable to said additional-identifier recording medium, when said complementary kit is used.

A kit may comprise one or more instruments, or one or more implants, or one or more instruments and one or more implants.

According to variants, said unique identifier consists of an optically recognisable code or is recorded in an electronic memory.

According to a first advantageous embodiment, the additional-identifier recording medium consists of a sheet bearing said unique identifier as well as areas for receiving the transferable identifier media and an area for receiving the patient code (8).

According to a second embodiment, the additional identifier recording medium consists of a computer file able to receive said unique identifier, the patient code and the transferable identifier media.

Preferably, the patient code is anonymous and non-unique.

According to a variant, the system according to the invention also comprises a database for recording the information relating to the manufacture, packaging and sterilisation of all the kits, the system comprising means for comparing the information issuing from the additional-identifier recording medium from the unique identifier associated with said media.

Advantageously, the kit comprising said unique identifier and said recording medium is the fitting instrument kit.

The invention also concerns a method for the traceability of a surgical instrumentation system consisting of a single-use instrument kit for fitting implants and at least one sterile implant kit, each of said kits comprising an identifier, characterised in that a patient code is recorded on an additional-identifier recording medium associated with one of said kits comprising a unique identifier, and in that furthermore at least one transferable identifier of at least one complementary kit is recorded on said additional-identifier recording medium at the time of use of said complementary kit.

According to one variant, the method comprises a step of transmitting, to a collecting database, said recording media at the end of the use of said system on a patient, and in that it comprises a step of editing a summary file from the collected data at least.

According to another variant, the method comprises a step of transmitting, to a collector for waste to be recycled, a traceability file grouping together the information coming from the recording file.

### BRIEF DESCRIPTION OF THE DRAWINGS

The present invention will be better understood from a reading of the following description referring to non-limitative examples of implementation illustrated by the accompanying drawings, where:

FIG. 1 shows a schematic view of kits for implementing the invention

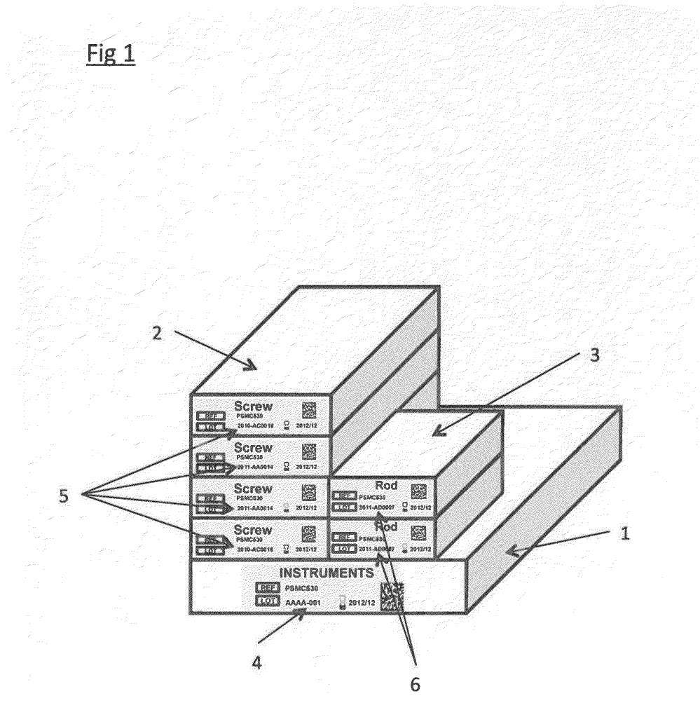

FIGS. 2 and 2bis show views of a kit after opening as well as the associated identifiers

FIG. 3 shows an example of an additional-identifier recording medium

FIG. 4 shows a view of the information flows when the invention is implemented.

### DETAILED DESCRIPTION OF A PREFERRED EMBODIMENT

The system according to the invention is composed of several single-use kits, for example kits comprising one or more fitting instruments packed in a sterile manner, and kits comprising one or more sterile prostheses or implants intended to be fitted with the instruments of the first kit.

### Context of the Use of Single-Use Kits

The prior art in the field of lumbar surgery of the spinal column consists of making implants and sets of instruments available to surgeons. Some companies specialising in the field offer implants packed in a sterile manner, ready for use.

Using sets of instruments reusable as required after decontamination and sterilisation is also known, which leads to many disadvantages:

the risk of contamination from one patient to another is very high, cleaning,

decontamination and sterilisation are steps that are very difficult to perform correctly because of the complex shapes, with numerous cavities on the instruments.

these steps are the responsibility of the hospitals and represent high costs, both human and material.

Loss, breaking, wear and damage to an instrument may cause disastrous consequences for the following patient, or even cause the surgery to be cancelled. Logistics also comprises numerous disadvantages, difficult and expensive both for the manufacturer and the hospital, and usual involves extensive inventories.

Rotations of kits between the hospital and the logistic service of the manufacturer are therefore very numerous, which significantly increases the risks of losses and errors.

The invention thus takes into account the safety aspect both for the patient and the surgeon, as well as the financial, human and material aspects for the hospital.

### Example of Kits for Spinal Surgery

The invention is described hereinafter with reference to a pedicle screw system described in the French patent application FR 09/06369.

The system is composed of several single-use kits:

the instrument kit, constituting the “single-use instrument kit for fitting implants (1)”, comprises all the instruments necessary for preparation and anchoring of the implant in the vertebrae and locking of the assembly. This kit protects the action of the surgeon through the use of new instruments at each intervention, removes any risks of nosocomial contamination and affords a significant reduction in public health costs;

sterile implant kits (2, 3) formed by:

an implant kit (2) comprises the pedicle screw and its cap, premounted on the fitting instruments, making use very intuitive. This kit is identified in several references: variants in diameter and screw length

a rod kit (3) comprising two connecting rods.

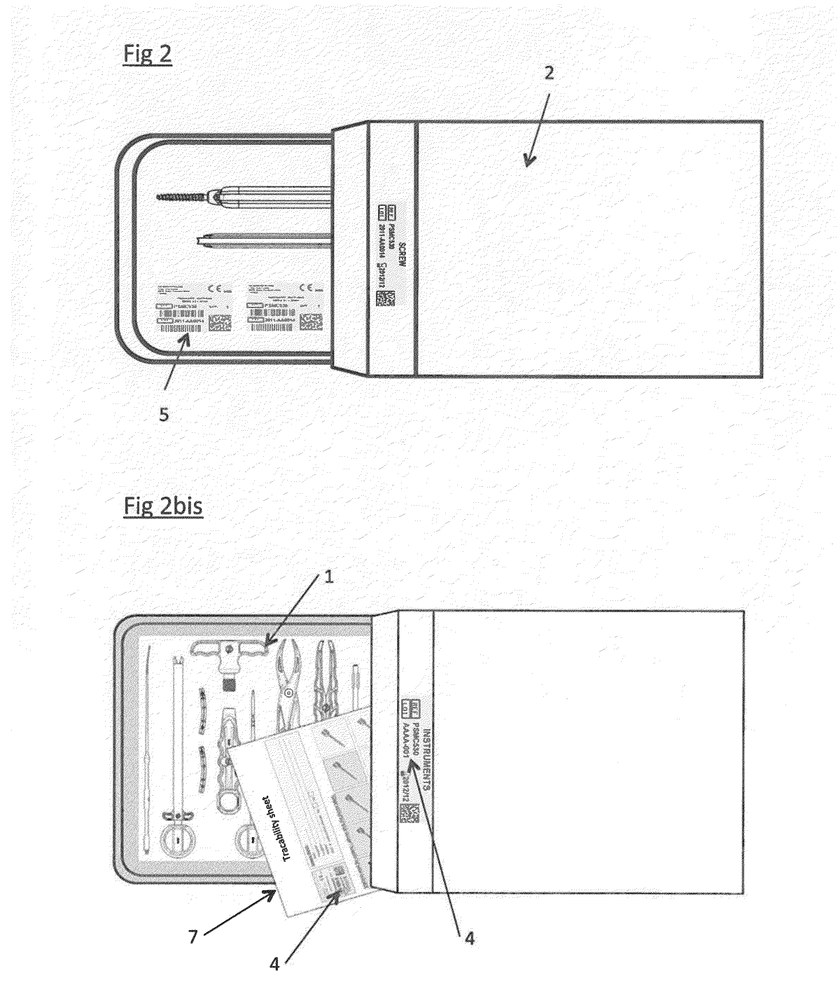

FIGS. 2 and 2bis show views of a kit respectively of implants (2) and instruments (1) after opening, as well the associated identifiers.

The instrument kits (1) contain a unique identifier (4) making it possible, when it is used in the operating theatre, to link it to a surgery (patient file), and other kits (2, 3) (rods or screws) used. This unique identifier (4) may be a serialised batch number, a bar code of the matrix code type “data matrix”, a code recorded in an electronic memory or any other means.

In each implant kit (2, 3), in accordance with directives, several transferable labels (5, 6) setting out the complete traceability of the content are available.

A recording medium consisting of a paper monitoring file is supplied in the instrument kit containing its unique identifier (4), and enabling all the “implant” labels (5) used during surgery to be produced, as well as codification of the associated patient (codification for confidentiality reasons). This codification is preferably of the “non-unique” type, for example the first three characters of the name of the patient, or even the date of the intervention without specification of the time. This guarantees the absence of any risk of breach of medical confidence.

The transferable labels (5) consist in the example described of self-adhesive labels on which an identifier is printed, for example the manufacturing batch number. This identifier is not necessarily unique, but it is obliged to be specific to the same component, issuing from the same manufacturing series.

### Additional-Identifier Recording Medium

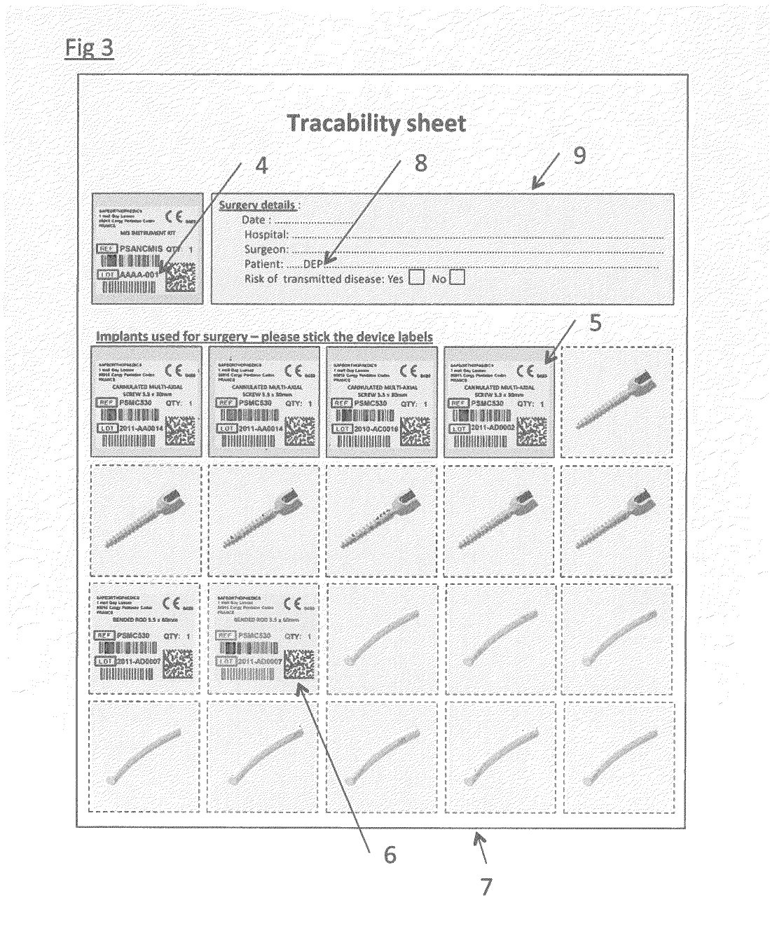

FIG. 3 shows an example of an additional-identifier recording medium (7).

This monitoring sheet (7), included in one of the kits, in the example described in the instrument kit (1), is used first by the hospital for completing the patient file and secondly to facilitate invoicing and restocking of kits and secondarily to enable the single-use instruments to be recycled.

It has areas intended for sticking labels (5, 6) removed from the implant kits (2, 3) during use of these.

On this monitoring sheet there is completed, by way of example, in order to enable:

anonymous identification of the patient, enabling the hospital to identify the patient without disclosing confidential information to the other participants in the procurement and possible recycling of the single-use instruments;

declaration of transmissible illness risk: the hospitals declares the risks of transmissible illnesses if the patient in infected by HIV, Creutzfeldt Jakob, etc;

the identifier of the other kits consumed.

This monitoring sheet is sent to an operator providing information to a database. The database is thus completed for triggering invoicing, reprocurement and recycling of the products as well as the transmission of a summary sheet containing all the traceability information, both that coming from the hospital and that coming from the manufacturing, packaging and sterilisation chain.

This monitoring sheet (7) may be directly completed via the interactive database.

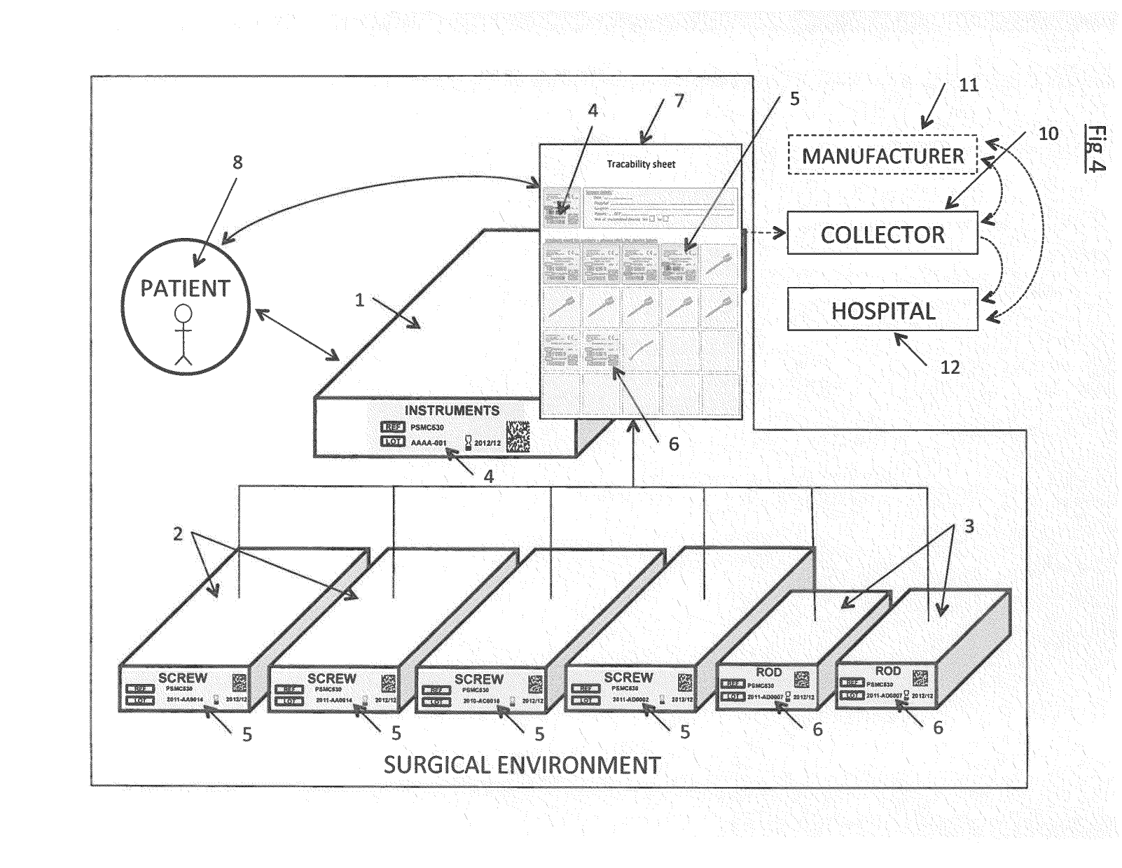

FIG. 4 shows a schematic view of the information flow.

The information reference element consists of the unique identifier (4) associated with the instrument kit (1), as well as with the recording medium (7) contained therein.

After the opening of the kit (1) for intervention on a patient, anonymous information (8) not directly linked to the patient is recorded on the recording medium (7), for example by writing a code (8) in the heading (9). This code (8) may consist of the first three letters of the name of the patient. This triplet is not unique, since several family names may begin with the same string of characters, for example the trigram “DEP” designating both “Claude DEPIPEAU” and “Marcel DEPUY”. The code formed by this trigram therefore does not by itself alone make it possible to identify the patient, and its transmission does not come within the scope of rules relating to medical confidentiality.

The hospital alone can reconstitute the link between the patient, his complete identity and the monitoring sheet (7). For this purpose, the hospital will associated the unique identifier (4) coming from the instrument kit (1) with the patient file in its possession, or will record on a duplicate of the monitoring sheet (7) the unique identifier of the patient, a duplicate that will remain under the sole control of the hospital.

In particular, the instrumentation kit (1) may comprise a self-adhesive label on which the unique identifier is printed or a transferable medium for facilitating the association of this identifier with the patient file.

The hospital will thus have available:

a patient dossier governed by medical confidentiality

an anonymous monitoring file

unequivocal information formed by the unique identifier and the non-unique code of the patient, enabling the patient file to be compared with the monitoring sheet (7).

On the other hand, the manufacturer does not have the patient file and the patient code affixed to the monitoring file (7) does under any circumstances enable him to identify the patient concerned.

During the intervention, the surgeon chooses from the implant kits (2, 3) the one or ones necessary for the surgical operation under way. Each of these kits (2, 3) comprises identifiers that may be common to several kits in the same series. For example, several rod kits comprise the same identifier, (XYZ) for those in the first series and (MNO) for those in the second series. These identifiers (5, 6) are moreover printed on a self-adhesive label contained in the implant packaging.

The label of the kit uses is detached from the kit and transferred to the monitoring sheet (7).

At the end of the intervention, the monitoring sheet (7) is transmitted to a collector (10) for example at the distributor or manufacturer of the kits, by fax, in the form of digital image files or by post, or by means of a mobile telephone equipped with a photographic means.

The collector (10) records all the information on the monitoring file (7) and where applicable compares it with the information coming from the manufacturer (11), and including where applicable the data relating to packaging and sterilisation.

The collector can then retransmit to the hospital (12) a traceability sheet identified by the combination of the unique identifier and the anonymous code (8) of the patient, and comprising information on the state of each of the tools and implants corresponding to the identifiers recorded on the monitoring sheet (7).

This traceability sheet can then be associated by the hospital with the patient file, which it alone is in a position to use and compare, since the hospital is the only one to hold the information enabling the non-unique code (8) to be associated with the secret identifier of the patient, by means of the unique identifier (4) of the instrument kit.

### Variant for Managing Recycling

At the present time, products for single use commonly used are treated as healthcare activity waste with risks of infections, disposed of in specific bins and then incinerated.

The packages of the medical devices according to the invention are designed so as to serve as a package for recycling.

After the surgery, the hospital declares the use of the device by entering the patient sheet in the database, which, as described previously, triggers reprocurement, but also makes it possible to decide on the possibility of recycling:

If the patient does not exhibit any transmissible illness (HIV, Creutzfeldt Jakob, etc.), the device is decontaminated by the hospital, reconditioned in the package. The manufacturer is identified either by a unique batch number and/or a brand (self-adhesive label, etc.), stored until the collection organised by the manufacturer.

At the same time, these products are inventoried in the database as products to be reprocessed; if the patient is ill (HIV, Creutzfeldt Jakob, etc.) or has symptoms without the diagnosis being determined, without traceability being possible just after surgery, the products are incinerated according to the hospital procedures.

The manufacture makes up recycling dossiers by editing from the database that contains the initial production information and the monitoring sheet and organises the collection of the parts. The products are physically sent to reprocessing sites responsible for cleaning and sorting the materials and reprocessing thereof, whatever they may be.

1-10. (canceled)

11. A system for the traceability of a surgical instrumentation system consisting of a single-use instrument kit for fitting an implant and at least one sterile implant kit, each of said kits comprising an identifier, and at least one of said kits containing a unique identifier, as well as a medium for recording additional identifiers, one of said additional identifiers being a patient code, collected when at least one of said kits is used, at least one other additional identifier being the identifier of at least one complementary kit comprising an identifier transferable to said additional-identifier recording medium, when said complementary kit is used.

12. A system for the traceability of a surgical instrumentation system according to claim 1, wherein said unique identifier consists of an optically recognisable code.

13. A system for the traceability of a surgical instrumentation system according to claim 1, wherein said unique identifier consist of a code recorded in an electronic memory.

14. A system for the traceability of a surgical instrumentation system according to claim 1, wherein the additional-identifier recording medium consists of a sheet bearing said unique identifier as well as areas for receiving a transferable identifier media and an area for receiving a patient code.

15. A system for the traceability of a surgical instrumentation system according to claim 1, wherein the additional-identifier recording medium consists of a computer file able to receive said unique identifier, a patient code and a transferable identifying media.

16. A system for the traceability of a surgical instrumentation system according to claim 1, further comprising a database for recording information relating to a manufacture, packaging and sterilization of the kits, and means for comparing the information issuing from the additional-identifier recording medium using the unique identifier associated with said media.

17. A system for the traceability of a surgical instrumentation system according to claim 1, wherein the at least one kit containing said unique identifier and said recording medium is a fitting instrument kit.

18. A method for the traceability of a surgical instrumentation system consisting of a single-use instrument kit for fitting implants and at least one sterile implant kit, each of said kits comprising an identifier, recording a patient code on an additional-identifier recording medium associated with one of said kits comprising a unique identifier, and recording at least one transferable identifier of at least one complementary kit on said additional-identifier recording medium at a time of use of said complementary kit.

19. A method for the traceability of a surgical instrumentation system according to claim 8, further comprising a step of transmitting said recording media to a collection database at an end of a use of said system on a patient, and a step of editing a summary file from a collected data.

20. A method for the traceability of a surgical instrumentation system according to claim 8, further comprising a step of transmitting, to a collector of waste to be recycled, a traceability sheet containing information coming from the recording media.
